(19) United States
(12) Patent Application Publication
(10) Pub. No.: US 2015/0018524 A1
(43) Pub. Date: Jan. 15, 2015

(54) WOUND SCREEN

(71) Applicant: Archimed LLP, Bedford (GB)

(72) Inventor: Paul James Davis, Felmersham (GB)

(21) Appl. No.: 14/374,079

(22) PCT Filed: Feb. 12, 2013
(86) PCT No.: PCT/GB2013/050314

§ 371 Date: Jul. 23, 2014

(30) Foreign Application Priority Data
Feb. 15, 2012 (GB) 1202614.2
Dec. 18, 2012 (GB) 1222771.6

(51) Int. Cl. A61L 15/42; A61L 15/44; A61L 15/32

(52) U.S. Cl. CPC A61L 15/425; A61L 15/32; A61L 15/44; A61L 2300/406; A61L 2300/204
USPC 530/377

(57) ABSTRACT

A binding screen comprising a gas-porous solid non-water-dispersible carrier, to which has been substantially irreversibly bound a compound having matrix metalloproteinase binding functionality, particularly doxycycline.

### TECHNICAL FIELD

The present invention relates to a binding screen for application to a wound of a human or animal, comprising a carrier, particularly for the reduction of proteolytic enzymes, particularly matrix metalloproteinases (MMPs), in a wound site to be treated.

### BACKGROUND AND PRIOR ART

There are various unmet needs in the area of wound care, and one of which is to do with the excessive quantities of proteolytic enzymes brought into non-healing wounds by infiltrating leukocytes.

There is a need for wound treatments that block or remove these enzymes, especially the MMPs, thus allowing the wound tissues to engage in the regeneration process more effectively. This unmet need is set to become even more prominent, as a result of the imminent introduction of new wound diagnostic technology that detects the presence of excess proteolytic enzymes in wound fluid.

Some products and technologies designed to block or remove excess proteolytic enzymes from wounds already exist, but they are widely recognised as being inefficient. The most widely used type of these products are based on denatured collagen. Collagen is, of course, one of the main targets of the MMPs. The idea is that the MMPs become drawn into attacking the collagen of the dressing, so distracting them from degrading the extracellular matrix and other beneficial molecules of the wound.

However, with the denatured collagen-type dressings it is claimed that the MMPs actually bind to the collagen of the dressing, but MMPs can cleave collagen, which would allow them to be released from the dressings and re-enter the wound cavity in an active form.

In addition, within the area of surgery, it has been suggested that doxycycline, a well-known inhibitor of MMPs, could be delivered to tissues damaged by surgical processes by releasably attaching doxycycline molecules to dissolvable sutures or solid tissue implants. The tissue implants would be coated with cleavable fibrinogen, and the doxycycline would be attached to that. Tissue remodelling activities involved in the healing process would dissolve the fibrinogen, thereby releasing the doxycycline to be free and active in the tissues. These approaches are disclosed in the following patent applications WO 2008/105732 and U.S. 2009/0177228

In the case of the doxycycline sutures, the sutures are designed to be dissolvable, and the attached doxycycline molecules are destined to be active when liberated from the solid structure, and not while it is attached. The sutures are designed to be a controlled delivery vehicle for doxycycline, leaving the active agent in the tissues. The sutures are designed to be placed inside tissues so any MMPs are left inside the body.

WO 2008/148174 discloses a hydrogel skin dressing to which is covalently bound a substance having MMP-binding functionality. The substance is intended to be retained within the hydrogel and removed from the wound site.

However with this approach, only the surface sites of the hydrogel near to the wound site are available to bind MMP and furthermore the hydrogel will interfere with the functioning of other dressing components as it stores and retains the wound fluid comprising MMPs.

There therefore remains a need for an improved method of inhibiting MMPs in wounds.

### DETAILED DESCRIPTION OF THE INVENTION

In a first aspect, the invention relates to a matrix metalloproteinase (MMP) binding screen, comprising a gas-porous solid non-water-dispersible carrier, to which has been substantially irreversibly bound a compound having and retaining MMP binding functionality.

The screen is therefore chemically bonded to the compound having MMP binding functionality, allowing no substantial release into the wound site. Thus, the compound having MMP binding functionality retains its ability to bind MMPs despite itself being substantially irreversibly bound to the carrier.

Additionally the screen is gas porous, which means that the wound fluid can pass into the interior of the screen quickly, making available all the internal MMP binding sites.

Furthermore, as the screen is gas-porous it can cooperate with other dressing components, as it allows wound fluid to pass through it.

Thus, the screen, when placed on or in a wound of a human or animal, binds any MMPs, which are then removed from the wound with the carrier when the screen is removed. The screen thus achieves its effect whilst remaining outside human tissues, ready for later removal.

The carrier material is a biocompatible carrier material, to which particular MMP-binding molecules are attached.

Some molecules with this ability are known and described in the literature, and these are often used as anti-inflammatory agents because of their ability to minimise some of the main pathogenic consequences of inflammation, caused by MMP activity. For the purpose of this invention, it is possible to use known inhibitor molecules if they have sufficient binding strength and compatible chemistry.

The compound having MMP binding affinity may be a member of the tetracycline group of antibiotics. Within this group doxycycline is the most preferred compound.

Alternatively, novel peptides that have MMP-binding affinity, but which are not cleaved by their enzymatic action, may be used. Such peptides can be discovered by means of random peptide-display libraries (e.g. bacteriophage peptide libraries), through phage-panning on immobilised MMP surfaces, using techniques well known in the art. In other peptide discovery techniques (e.g. Pepscan techniques) peptides may be immobilised and arrayed on the surface. An aqueous solution of the MMPs can be applied to these immobilised peptides, after which the surface is tested for the presence of captured (retained) MMP, for example, by the use of a labelled antibody that recognises one or more type of MMP on the surface.

Whichever MMP-binding compound is used the chemical linkage to the carrier material is substantially irreversible, and the MMP-binding compound becomes incorporated into the chemical structure of the carrier. Effectively, as the chemical linking takes place, the MMP-binding compound, e.g. doxycycline, becomes a new chemical entity formed with the carrier material. The carrier material and the MMP-binding compound are transformed into a composite MMP-binding solid substance.

The carrier material, such as a cellulose pad or gauze preferably has a high surface area so that a high density of MMP-binding groups can be attached.

The composite material is designed to be placed on a wound or in a wound cavity, where it binds MMP molecules substantially irreversibly and hold them in place (and inactive), until it is removed at dressing change.

A number of different carrier materials are suitable, providing that the material has chemical functional groups that can be derivatised to make them reactive with appropriate functional groups on the MMP-binding compound, or vice versa.

Cellulose structures, such as cotton gauze or cotton wool, are especially suitable. A variety of non-woven fabrics are suitable. These are made from long fibers, bonded together by chemical, mechanical, heat or solvent treatment to yield materials which are neither woven nor knitted. Non-woven structures in a format similar to structures used as filters can be adapted as the carrier material. Nylon fabrics can also be chemically processed to carry the MMP-binding molecules in the wound. The choice of carrier material is guided by criteria well known in the wound-care field.

In particular, the material must be biologically inert (not eliciting any inflammatory reactions and not possessing any toxicity), it must retain its structural integrity within the wound environment, it must be compatible with (and not impede) other wound dressings designed to interact with the wound (e.g. foams, hydrocolloids, hydrogels etc.) and it must be sufficiently conformable to lay in contact with complex wound surfaces while remaining sufficiently structured to be easily applied and removed.

In addition the screen is desirably non-swellable, so that any absorbed wound fluid merely passes through the screen, without necessarily being retained therein.

The chemistry of attachment of MMP-binding compound must be able to proceed without impairing the ability of MMP-binding compound to bind to, and deactivate, MMPs.

In one preferred route, the MMP-binding compound is derivatised by introducing an amine group removed from the MMP-binding functional region. This can be carried out, for example, by nitration followed by hydrogenation.

The provision of an accessible primary amine group on the MMP-binding compound (at a site on the molecule sufficiently distant from the MMP-binding motif to not impair binding) enables a range of possible coupling chemistries involving aldehydic carbonyl groups and carboxyl groups on the carrier molecules. It is also possible to provide a carbonyl or carboxyl group on the MMP-binding molecule to react with primary amines on the carrier. Alternatively, a thiol group can be provided, to allow conjugation via a disulfide bond a thiol group on the carrier.

The invention will now be illustrated by the following non-limiting example:

### EXAMPLE

The molecular structure of doxycycline is shown below:

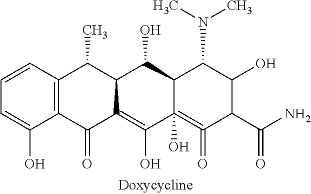

To incorporate doxycycline into the compound structure of a carrier material, doxycycline must first be converted into a chemically active derivative. This is achieved by generating a reactive amine group at position 9 through which further covalent coupling can be carried out.

The doxycycline is derivatised at this position, to allow the main structure of the molecule to remain intact, since it is believed that this main structure confers the antibiotic activity. The carbonyl-linked amide group at position 2 is, therefore, not used for the derivatisation steps.

The derivatisation is performed as follows.

### Step 1 (Nitration). Compound A to Compound B

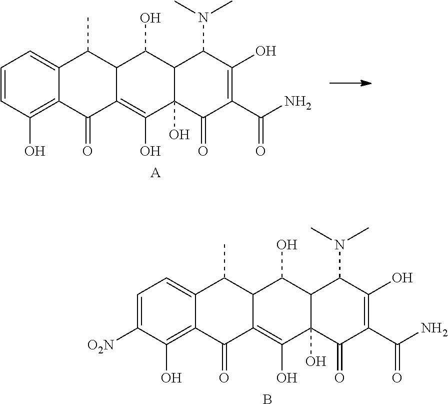

### Step 2 (Hydrogenation). Compound B to Compound C

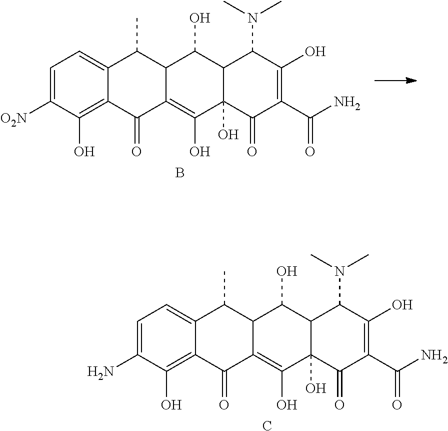

The amine group at position 9 can then be used for coupling reactions to any suitable carrier substrate, for example, cellulose partially oxidised by reaction with periodate, or carboxyl bearing surfaces such as certain types of nylon, using hetero-bifunctional cross-linking reagents.

Another approach to matrix coupling is to further modify compound C via biotinylation. Biotin is covalently coupled to the free amine at position 9, to yield compound D, as shown in the following schematic:

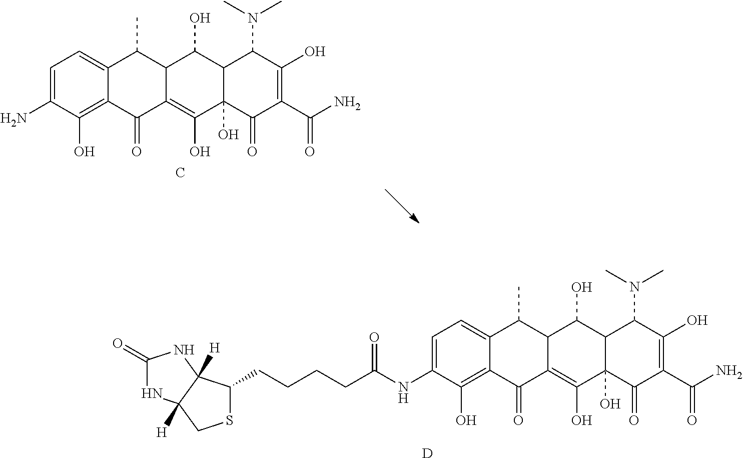

A suitable carrier substrate, can then be modified with avidin or streptavidin (or any other avidin derivative or analogue) via known covalent coupling methods, and the modified substrate and compound D mixed together to allow the biotin and avidin to bind together. The avidin-biotin complex is well characterised and is accepted to be one of the strongest non-covalent complexes known, with a dissociation constant (K_{D}) of ˜10⁻¹⁵M.

A second example of a coupling reaction, is to covalently attach compound C to a suitable substrate. A suitable substrate could be a gauze fabric, prepared from cotton. The cotton is treated with sodium periodate, which will oxidise the β-D-glucopyranose residues to give free aldehydes. Compound C is mixed with the activated cotton, and the aldehydes will under go nucleophilic attack by the free amine (at position 9) to form carbinolamines. The mix is then subsequently treated with a reducing agent such as sodium borohydride which will both reduce any unreacted free aldehydes, thus preventing any unwanted further reactions, and also reduce the unstable carbinolamines to the more stable alkylamines.

The resultant, chemically modified mesh has the ability to bind and neutralise MMPs present in wound fluid with which the mesh is placed in contact.

1. A matrix metalloproteinase binding screen, comprising a gas-porous solid non-water-dispersible carrier, to which has been substantially irreversibly bound a compound having and retaining matrix metalloproteinase binding functionality.

2. A method of preparing the screen according to claim 1, wherein a compound having matrix metalloproteinase binding functionality is converted into a chemically active derivative having a chemically active site and leaving the matrix metalloproteinase binding functionality substantially unaffected, which compound is then brought into contact with the carrier and is substantially irreversibly bound thereto via the chemically active site.

3. The method according to claim 2, wherein the compound having matrix metalloproteinase binding functionality is converted into a chemically active derivative by introducing an amine group.

4. The method according to claim 3, wherein the carrier comprises cotton having β-D-glucopyranose residues, which is treated with sodium periodate, which oxidises the β-D-glucopyranose residues to give free aldehydes, the amine derivative then being mixed with the treated cotton to form a mix, so that the aldehydes undergo nucleophilic attack by the free amine to form carbinolamines, the mix then subsequently being treated with a reducing agent which both reduces any unreacted free aldehydes, thus preventing any unwanted further reactions, and also reduces carbinolamines to alkylamines.

5. The method according to claim 3, wherein the amine functionality is further converted by biotinylation to add a biotin, and then brought into contact with the carrier, which has been modified with an avidin derivative or analogue, to allow the biotin and avidin to bind together.

6. The screen according to claim 1, wherein the compound having matrix metalloproteinase binding functionality comprises a tetracycline antibiotic.

7. The screen according to claim 6, wherein the tetracycline antibiotic is doxycycline.

8. The screen according to claim 1, wherein the screen is non-swellable.

9. The screen according to claim 1, wherein the carrier comprises cotton and alkylamines.

10. The method according to claim 2, wherein the compound having matrix metalloproteinase binding functionality comprises a tetracycline antibiotic.

11. The method according to claim 10, wherein the tetracycline antibiotic is doxycycline.

12. The method according to claim 2, wherein the screen is non-swellable.
